(19) United States
(12) Patent Application Publication
(10) Pub. No.: US 2022/0208329 A1
(43) Pub. Date: Jun. 30, 2022

(54) PRIME DIFFERENTIATION IN MEDICATION DELIVERY SYSTEM

(71) Applicant: Companion Medical, Inc., San Diego, CA (US)

(72) Inventors: Michael Mensinger, San Diego, CA (US); Jack Pryor, Ladera Ranch, CA (US); Angela Gaetano, San Diego, CA (US); Sean Saint, San Diego, CA (US); Andrew Childs, San Diego, CA (US); Haripriyan Hampapuram, Carlsbad, CA (US); Arnold Holmquist, Dana Point, CA (US); Heung Yong Im, La Jolla, CA (US); Madison Smith, San Diego, CA (US)

(21) Appl. No.: 17/604,074

(22) PCT Filed: Apr. 17, 2020
(86) PCT No.: PCT/US2020/028809

§ 371 Date: Oct. 15, 2021

Related U.S. Application Data
(60) Provisional application No. 62/993,826, filed on Mar. 24, 2020.
(60) Provisional application No. 62/835,390, filed on Apr. 17, 2019.

(51) Int. Cl. G16H 20/13

(52) U.S. Cl. CPC G16H 20/13

(57) ABSTRACT

Systems, devices, and techniques are disclosed for differentiating a therapeutic dose and a priming event to properly administer medication to patients. In one example aspect, a method for differentiating therapeutic doses and priming events in administering a medication to a patient includes determining a time window for monitoring dispensing of the medication by an injection pen device. Multiple dispense events each releasing an amount of the medication occur in the time window. The method also includes classifying dispense events prior to a last dispense event in the time window as priming events or therapeutic doses and designating the last dispense event in the time window as a therapeutic dose for facilitating calculation and tracking of a dosage amount for the patient.

### CROSS-REFERENCES TO RELATED APPLICATIONS

The present application claims priority to U.S. Provisional Application Ser. No. 62/835,390 filed on Apr. 17, 2019 and entitled “Prime Differentiation in Medication Delivery System,” and U.S. Provisional Application Ser. No. 62/993,826 filed on Mar. 24, 2020 and entitled “Prospective Prime Differentiation in Medication Delivery System,” the entire contents of each of which are incorporated by reference into this disclosure for all purposes.

### TECHNICAL FIELD

This patent document relates to medication administering and tracking systems, devices, and processes.

### BACKGROUND

Diabetes mellitus, also referred to as diabetes, is a metabolic disease associated with high blood sugar due to insufficient production or use of insulin by the body. Diabetes is widely-spread globally, affecting hundreds of millions of people, and is among the leading causes of death globally. Diabetes has been categorized into three categories or types: type-1, type-2, and gestational diabetes. Type-1 diabetes is associated with the body's failure to produce sufficient levels of insulin for cells to uptake glucose. Type-2 diabetes is associated with insulin resistance, in which cells fail to use insulin properly. The third type of diabetes is commonly referred to as gestational diabetes, which can occur during pregnancy when a pregnant woman develops a high blood glucose level. Gestational diabetes can develop into type-2 diabetes, but often resolves after the pregnancy.

Various diseases and medical conditions, such as diabetes, require a patient to self-administer doses of a fluid medication. Typically, when administering a fluid medication, the appropriate dose amount is set and dispensed by the patient using a syringe, a pen, or a pump.

A medicament pen is a device that can be used to inject a quantity of a medicine (e.g., single or multiple boluses or doses of the medicine) into a user's body, where more than one dose can be stored in a medicine cartridge contained in the pen device. Pens offer the benefit of simplicity over other methods of delivery, such as syringe or pump-based methods.

For medicament pens (as well as other injectors of other types and for other therapies), a user typically prepares the pen to remove air and confirms correct operation prior to injecting a therapeutic dose. This preparation process is also referred to as “priming” the pen or a priming event. The priming process can be dependent on user behavior and the state of the system—sometimes multiple priming events are needed before a therapeutic dose is taken; sometimes multiple therapeutic doses may be taken in a row; sometimes a device may be primed but no therapeutic dose is dispensed; and sometimes a user may neglect to prime the device altogether. To accurately track dosage received by the patient, it is important for the device to differentiate between priming events and therapeutic doses so that only the therapeutic doses are used for calculating and adjusting the user's medication dose.

### SUMMARY

The disclosed embodiments relate to an intelligent medicine administering and tracking system. One example system includes a pen device in wireless communication with a mobile computing and communication device of a patient user, also referred to as the user's companion device. The pen device is operable to select, set and/or dispense a dose of the medicine for dispensing. In some implementations, the companion device comprises a smart phone, tablet, and/or wearable computing device, such as a smart watch, smart glasses, etc. In some implementations, the companion device is in communication with other computing devices, such as a laptop and/or desktop computer, a smart television, or network-based server computer. The pen device and/or companion device includes a health management software application (“app”) that may be associated with the pen device and other smart health management accessories/devices of the intelligent medicine administering system. In one example embodiment, the pen device and/or the companion device is able to distinguish between a priming event or “prime” and a therapeutic dose or “dose” when medicine is dispensed from the pen device. In one embodiment, the health management app includes an adaptive dose calculator and/or decision support modules to suggest the dose for the patient and provide control over several functionalities of the pen device.

The health management app of the companion device associated with the pen device provides a user interface to allow the user to manage his/her health-related data. In some implementations, for example, the health management app can be configured to control some functionalities of the pen device. In some implementations, for example, the health management app provides an interactive user interface to allow a user to manage settings of the pen device, and settings for the companion device (e.g., smart phone, tablet, or wearable computing device) that can affect the functionality of the system. In implementations, for example, the companion device is an independent portable device that the user may carry on his/her person. In example embodiments of the independent portable companion device, the companion device includes a data processing unit, wireless communication unit to allow the device to communicate with the pen device, and a display unit.

In some implementations, for example, via the user interface of the health management app, the companion device allows the patient to browse a list of previous doses, to view an estimate of current medicament active in the patient's body (“medicament on board”) based on calculations performed by a medicine calculation module of the health management app, and/or to utilize a dose determination module of the software application to assist the patient regarding dose setting information on the size of the next dose to be delivered. For example, the patient may enter carbohydrates to be eaten and current blood sugar, and the companion device would already know insulin on board. Using these parameters, a suggested medicine dose (e.g., such as insulin dose), calculated by the dose determination module, may be determined.

The embodiments of the disclosed technology, among other features and benefits, provide multiple methods for a pen device or smart injector and/or health management app to differentiate between therapeutic doses and prime events. In some scenarios, a method for evaluating or detecting medication doses versus primes may be ambiguous, and in these scenarios, it can be useful for a datalogger component to record or otherwise retain the information regarding the ambiguity, so it may prompt the user for manual confirmation. Typically, overdosing on insulin is more harmful than taking too little, so in the event of an ambiguity it is safer to assume that a dispense event was taken as a dose. For example, in subsequent calculations, the presence of an ambiguity may be taken into account to artificially decrease doses the user will take. In contrast, mistaking a dose as a prime would cause the system to under-estimate the amount of insulin a user has taken, causing subsequent calculations to recommend too much insulin to be dispensed.

A user typically primes the dispensing device before applying a therapeutic dose, possibly multiple times. Accordingly, by way of example, the intelligent medicine administering system may evaluate a pattern or sequence of dispense events within a given time window, designating the last event in the pattern as a therapeutic dose and designating events prior to the last event in the pattern as priming events. However, several scenarios are described herein by way of example that occur during normal use of the intelligent medicine administering system for which the last event in a sequence of dispensing events is not actually the therapeutic dose, including scenarios in which one or more therapeutic dose events occur prior to the last event in a sequence of dispensing events. The intelligent medicine administering system is configured to recognize and account for these scenarios when designating a dispensing event as prime or dose.

In some embodiments, a manually-actuated injector pen device that senses advancement of the cartridge plunger (or detects mechanism movement related to plunger advancement, such as knob or screw rotation) may differentiate between pauses in a user's movement during a single injection event and/or separate injection events. The system may use a preset timeout threshold, such as one second, so that movements within the preset timeout threshold are considered to be a single dispense event, and movements outside of the timeout threshold are considered separate dispense events (e.g., a prime and a dose). However, different users dispense at different rates and with different pauses between dispense events, and therefore a default timeout threshold may not be sufficient for all users. Thus, in some embodiments, the intelligent medicine administering system may be configured to determine a user-specific timeout threshold using one or more of the several methods disclosed herein.

While priming behavior may vary from user to user, any single user often exhibits consistent priming behavior. Thus, analysis of a user's specific dispensing history may inform the determination of whether a questionable dispensing event is a prime or a dose event. By way of example, the intelligent medicine administering system of the present disclosure may be configured to monitor and analyze a specific user's dispensing activity history to identify (1) the user's typical dispense amounts in prime and dose event, and (2) the user's typical priming behavior. This data, by itself or in combination with other methods disclosed herein, may help inform the prime/dose determination.

In some instances, a dispense event may be inadvertent. Logging these inadvertent movements as therapeutic doses may create inaccurate records and lead to inaccurate calculations in the future. Thus, if the intelligent medicine administering system detects a dispense event that may be inadvertent, the system may disregard such a dispense event, record the event as a null event, or prompt the user to confirm whether the dispense event is valid (e.g. dose or prime).

In some embodiments, manual input from the user may provide accurate information and can help detect infrequent errors even when automatic dose/prime detection is available.

In some embodiments, the intelligent medicine administering system may display the dose amount detected by the pen device in real-time, allowing the user to verify that their actions were correctly interpreted by the system, and providing the user with an opportunity to correct the data logs if necessary.

In some embodiments, the pen device may include an accelerometer configured to sense the difference between a pen device held freely in the air and a pen device held steady against a user's skin, and may distinguish between a prime dispensing event and dose dispensing event accordingly.

In some embodiments, the system may require that the user primes the pen device prior to enabling use of the dose calculator (or prior to displaying a calculated result on the display unit of the companion device). In such embodiments, the system may consider dispense events occurring before dose calculator usage to be priming events and dispense events occurring after the dose calculator usage to be therapeutic doses.

In some embodiments, the pen device may be configured to force priming by mechanically locking the dispense mechanism unless and until a dispense event occurs while the distal or dispensing end of the pen device is pointed upward.

In some embodiments, the intelligent medicine administering system may automatically prime the pen device so that the user does not need to dispense a separate priming event.

In some embodiments, the intelligent medicine administering system may be configured to detect whether the pen device is near to or in contact with skin to determine if a dispense event is a therapeutic dose or a prime event.

In some embodiments, the pen device may have a shroud around the needle that allows liquid to exit for priming but guards against accidental contact of the needle with the user or with other objects in the environment. The pen device may include a switch configured to detect translation of the shroud, thereby exposing the needle and indicating that the dispense event is a therapeutic dose.

In some embodiments, a pressure sensor or vibration sensor provided on the needle or needle attachment may detect forces and vibration exerted by the user's skin on the needle. In response to the presence of force or vibration in the needle as detected by the pressure or vibration sensor, the system may record an associated dispensing event as a therapeutic dose.

In some embodiments, a capacitive sensor may be electrically coupled to the needle to electrically detect skin contact.

In some embodiments, a coil may be positioned around the needle to measure its inductance, which changes once the needle makes contact with skin.

In some embodiments, two or more electrical contacts positioned at the needle end of the pen device (one of which may be the needle itself) may sense when an electrical circuit is completed (closed), indicating that the contacts are touching the user's skin and the system may designate the dispense event as a therapeutic dose.

In some implementations, the pen device may have a thermal sensor at the needle end, configured to detect body heat of a user.

In some embodiments, a depth sensor (e.g. a laser, optical, or ultrasonic) may be oriented toward the needle end of the pen device. When the depth sensor detects a surface nearby (e.g., within the length of a typical pen needle, such as 5 mm) this indicates that the needle is injected into the surface and the system may identify the dispense event as a therapeutic dose.

In some embodiments, the intelligent medicine administrating system and/or pen device may use a single method or multiple methods disclosed herein in combination to differentiate a therapeutic dose from a priming event. For example, the system may utilize pattern recognition, a depth sensor, and an accelerometer. In some implementations, the system may aggregate different inputs probabilistically, such as with a voting scheme, so that the majority of multiple inputs are accepted as truth. In some embodiments, the system may conservatively assume that any of the inputs indicating a therapeutic dose is correct. In this mode, the system may only identify a dispense event as a priming event if all inputs indicate it as such. In some implementations, if all inputs do not agree and corroborate the dose/prime designation, the user is prompted by the system to provide confirmation. In some embodiments, the system may use a primary method to determine therapeutic/priming event designation. When the result of the primary method is ambiguous, the system may use a secondary method to assist the determination.

In some instances users may use a dose calculator to help them ensure they are injecting the correct therapeutic dose. By way of example, a dose calculator is a tool for people with diabetes to calculate a rapid-acting insulin dose, based upon planned carbohydrate intake, current glucose level and insulin still active from prior injections. The output of a dose calculator is a dose recommendation displayed by the intelligent medicine administering system on the user interface of the companion device and/or pen device. For a good experience, the dose recommendation should be displayed until the user injects that dose, and then the system may update the user interface of the companion device to remove the recommendation and optionally show the dose has been taken, giving positive confirmation of the action. Moreover, if a user is administering a multi-part dose, the recommendation may be updated in real time to inform the user how much of the dose recommendation is left to inject on the next injection.

Various methods are described herein by which the intelligent medicine administering system may prospectively distinguish between a prime dose and a therapeutic dose and as a result remove the dose recommendation from the user interface of the companion device (also referred to herein as “recommendation dismissal”). In some embodiments, the system may incorporate learned prior priming and use patterns, specific to a user and customized to the user's own recent behavior, to determine whether a dose was therapeutic to inform the appropriate timing of the recommendation dismissal. In some embodiments, the system may use confidence scoring to determine whether a dose was therapeutic. In some embodiments, the system may incorporate user interactions to improve the user experience. In some embodiments, the system may update the dose recommendation in real time to inform users administering therapeutic doses over several injections.

For the purposes of this disclosure, the “insulin recommendation” is the last dose amount recommended by the system dose calculator before the user begins dispensing insulin. An “episode” is the collection of dose activity (e.g., prime and/or therapeutic) within a predetermined period of time following the insulin recommendation. The system determines the episode start time by referencing the stored datetime value of the insulin recommendation.

In some embodiments, the system may determine the user's rolling prior prime behavior by analyzing the user's prime activity from within a set of episodes starting within a predetermined period of time prior to the start time of the current episode. The predetermined period of time may be any period of time that is sufficient to establish a pattern of behavior by the user. By way of example, the predetermined time period may be one week, resulting in the system determining a rolling week prime behavior. Once the set of episodes has been determined, the system uses stored data from those episodes to calculate a representative or typical prime count value, a representative or typical prime amount value, and a representative dose duration value, all specific to the user, that the system will later use to determine the appropriate timing of recommendation dismissal.

In some embodiments, the system may determine the representative or typical prime count value using any of a variety of methods, including but not limited to finding a median prime count value based on the user's recorded recent behavior, finding a mean prime count value based on the user's recorded recent behavior, prompting the user to select a prime count to be used, finding a mean prime count value across a population of a subset thereof, relying on a default prime count value preselected in the software app, and the like.

In some embodiments, the system may determine the representative or typical prime amount value using any of a variety of methods, including but not limited to finding a median prime amount value based on the user's recorded recent behavior, finding a mean prime amount value based on the user's recorded recent behavior, prompting the user to select a prime amount to be used, finding a mean prime amount value across a population of a subset thereof, relying on a default prime amount value preselected in the software app, and the like.

In some embodiments, the system may determine the representative dose duration time value using any of a variety of methods, including but not limited to calculating a representative dose duration time value based on the user's recorded recent behavior (e.g., finding a median value, mean value, or selecting a value on a bell-curve distribution in the third or fourth quartile, etc.), prompting the user to select a dose duration time value to be used, finding a mean dose duration time value across a population of a subset thereof, relying on a default dose duration time value preselected in the software app, and the like.

In some embodiments, the system may remove or dismiss the dose recommendation from the user interface screen of the companion device and/or pen device when the system determines that the sum value of the doses since the insulin recommendation time reaches or exceeds the following threshold:

(representative prime amount)*(representative prime count)+(insulin recommendation)−(dialing error correction value)

By way of example the dialing error correction value may vary depending on the increment size of the pen device in use (e.g., 0.5 for a half-unit increment pen device, 1.0 for a one-unit increment pen device, etc.).

In some embodiments, the system may remove or dismiss the dose recommendation from the user interface screen of the companion device and/or pen device when the system determines that the dose has a volume value greater than or equal to a predetermined number of units.

In some embodiments, the system may remove or dismiss the dose recommendation from the user interface screen of the companion device and/or pen device when the system determines that the user's representative dose duration time from the previous week, and no more than two minutes, has passed since the time of the first dose.

In some embodiments, the system may remove or dismiss the dose recommendation from the user interface screen of the companion device and/or pen device when the system determines that the insulin recommendation is 0.5, the user's median prime count=0, and the first positive-amount dose occurs.

In some embodiments, the system may remove or dismiss the dose recommendation from the user interface screen of the companion device and/or pen device when the system determines that the insulin recommendation is 0.5, the user's median prime count>0, and the second positive-amount dose occurs.

In some embodiments, the system may remove or dismiss the dose recommendation from the user interface screen of the companion device and/or pen device when the system determines that the user manually enters a dose value.

In some embodiments, if a user manually changes a dose type to “prime”, the system may re-display the dose recommendation on the user interface, exclude the dose manually changed to “prime,” and use one of the following to determine recommendation dismissal timing: (i) insulin recommendation—0.5, (ii) dose has a volume value greater than or equal to 4 units (for example), or (iii) the user's representative dose duration time from the previous week (and no more than two minutes) has passed since the time of the first dose.

In some embodiments, the system may use confidence scoring to determine whether a dose was therapeutic. In some embodiments, understanding the distributions of the users' prime behaviors can lead to a confidence scoring method for new dose interactions in the application. In some embodiments, other features such as paired recommendation amount may also influence the confidence score.

In some embodiments, the system may require a minimum confidence score to designate as a dose amount value to be therapeutic.

In some embodiments, the system may establish a tunable indeterminate zone in between a confident therapeutic dose amount value threshold and confident prime dose amount value threshold. If confidence scores fall within the tunable indeterminate zone, the system may implement one or more actions, including but not limited to displaying the dose recommendation on the user interface screen until a predetermined user episode duration (e.g., average, median, or a percentile value such as 90th) has passed, displaying the recommendation on the user interface screen 36 until a predetermined fixed amount of time has passed, dismissing the dose recommendation from the user interface screen 36, designating the dose as a therapeutic dose, designating the dose as a prime dose, displaying on the interface screen an indication of unrecognized dose type and prompting the user to designate the dose manually.

In some embodiments, particularly for users that don't prime the pen device, or for inconsistent users that may cause inappropriate recommendation dismissals, the system may flag the user as potentially benefiting from additional education, and activate an interactive training module to educate the user to practice consistent priming.

In some embodiments, the system may update the dose recommendation in real time to inform users administering therapeutic doses over several injections.

By way of example, data that the system may include in the confidence analysis include (but are not limited to): glucose response after dose, timing of the dose, number of doses within a time, deviation from normal dosing pattern, user feedback from the system designation fed back into the confidence score analysis, and additional sensor data available from the pen device such as orientation, force, speed of delivery, absolute plunger position sensor, etc.

In some embodiments, a combination of the above data points can be used in a time series anomaly detection system that indicates concern whenever the dose is significantly different from the otherwise normal predicted dose.

In some embodiments, the system may be configured to display dose recommendations for a predetermined amount of time (e.g., 1 minute, 2 minutes, etc.) after every dose, such that the dose recommendation will be displayed past prime doses and until the therapeutic dose for most users.

In some embodiments, the system may personalize this amount by measuring the episode duration from past dosing episodes as defined as the time from the first prime dose to the actual therapeutic dose (the last dose event in the sequence).

In some embodiments, the system may be configured to designate all doses having a dose amount value less than a predetermined dose amount value as prime initially, anticipating another dose to follow. If after a timeout, no dose is taken after the last dose, then the system may change the designation of the last dose or doses in a multi-part dose episode from prime to therapeutic. The last dose or several doses is typically the therapeutic injected dose.

The following provides some example embodiments of the disclosed technology.

Embodiment 1 is a method for determining an appropriate time duration to display a therapeutic dose recommendation associated with a current dispense event from a medicine dispensing device, comprising: (1) displaying, on a display device, a dose recommendation comprising a recommended therapeutic dose amount of medicine for a user of a medicine dispensing device; (2) analyzing use data pertaining to at least one of past and present dispense events for a particular user; and (3) removing the dose recommendation from the user interface based on the analysis of the use data.

Embodiment 2 is the method of embodiment 1, wherein the use data comprises volume data from the last dispense event, and the volume of the last dispense event exceeds a preset threshold value.

Embodiment 3 is the method of embodiments 1 or 2, wherein analyzing use data comprises: (1) identifying a set of episodes occurring within a predetermined period of time prior to the current episode, wherein an episode comprises a collection of prime dispensing activity and therapeutic dose dispensing activity occurring within a defined period of time following the display of the dose recommendation; (2) determining a representative prime count value based upon a count of prime events per episode within the identified set of episodes; (3) determining a representative prime amount value based upon a volume of dispensed medicine in prime events per episode within the identified set of episodes; (4) determining a representative dose duration time value based upon dose duration time values per episode within the identified set of episodes; and (5) calculating an appropriate timing for dose recommendation dismissal using at least one of the representative prime count value, the representative prime amount value, and the representative dose duration value.

Embodiment 4 is the method of any of embodiments 1 through 3, wherein analyzing use data comprises: (1) calculating a sum value of dispense events occurring since the display of the dose recommendation, wherein a dispense event comprises any volume of medicine ejected from the medicine dispensing device; and (2) comparing the calculated sum value of dispense events to a user-specific threshold value.

Embodiment 5 is the method of any of embodiments 1 through 4, wherein the user-specific threshold value is calculated using: (representative prime amount)*(representative prime count)+(dose recommendation)−(dialing error correction value)

Embodiment 6 is the method of any of embodiments 1 through 5, wherein the step of removing the dose recommendation from the user interface based on the analyzed use data comprises: upon determining that the calculated sum value of dispense events at least one of meets and exceeds the user-specific threshold value, removing the displayed dose recommendation from the display device.

Embodiment 7 is the method of any of embodiments 1 through 6, wherein the predetermined period of time comprises a length of time sufficient to establish a pattern of behavior of a user of the medicine dispensing device.

Embodiment 8 is the method of any of embodiments 1 through 7, wherein the predetermined period of time comprises one week.

Embodiment 9 is the method of any of embodiments 1 through 8, wherein analyzing use data pertaining to at least one of past and present dispense events for a particular user comprises: (1) analyzing a set of past prime dispense events to calculate a confidence score that a given dispense event is a prime event; and (2) analyzing a set of past therapeutic dispense events to calculate a confidence score that a given dispense event is a therapeutic dose event.

Embodiment 10 is the method of any of embodiments 1 through 9, wherein the step of removing the dose recommendation from the user interface based on the analyzed use data comprises: upon determining that the current dispense event has a volume that at least one of meets and exceeds a preset confidence score threshold value, removing the displayed dose recommendation from the display device.

Embodiment 11 is the method of any of embodiments 1 through 10, wherein the display device comprises at least one of the medicine dispensing device and a mobile communication device associated with the medicine dispensing device.

Embodiment 12 is a method for differentiating between a therapeutic dose and a priming event in dispensing medication by a medication dispensing device, comprising: (1) analyzing a plurality of dispensing events that occur in a sequence, the sequence including at least a first dispensing event and a last dispensing event, each dispensing event comprising a dispensed amount of medication from the medication dispensing device; (2) classifying at least one of the dispensing events occurring prior to the last dispensing event as a therapeutic dose dispensing event based on at least one of: (a) the dispensed amount has a volume that is above a predefined threshold volume; (b) the dispensed amount has a volume that is equal to a maximum volume capacity of the dispensing device; and (c) the dispensed amount exhausts the supply of medicine contained within the dispensing device.

Embodiment 13 is a method for differentiating therapeutic and priming events in administering a medication by a medication injection device, comprising: (1) analyzing a plurality of dispensing events that occur in a sequence within a preset time period and within a dose window; and (2) classifying at least one of the dispensing events as a therapeutic dose dispensing event based on one or more criteria.

Embodiment 14 is the method of embodiment 13, further comprising: (1) estimating a duration of a single dispensing event in the sequence to differentiate a single dispensing event from multiple consecutive dispensing events in the sequence using a variable time threshold; and (2) deriving a time window based on the duration of the single dispense event.

Embodiment 15 is the method of embodiments 13 or 14, wherein the variable time threshold is adjusted based on past dispensing events of a user of the medicine injection device.

Embodiment 16 is the method of any of embodiments 13 through 15, wherein the variable time threshold is adjusted based on a speed of movement when a user manipulates the medication injection device for dispensing a therapeutic dose or dispensing a prime dose.

Embodiment 17 is the method of any of embodiments 13 through 16, wherein the classifying comprises designating a dispensing event as the therapeutic dose dispensing event upon determining that an amount of the medication released in the dispensing event exceeds a predefined threshold.

Embodiment 18 is the method of any of embodiments 13 through 17, wherein the predefined threshold is determined based on past dispensing events of the patient.

Embodiment 19 is the method of any of embodiments 13 through 18, wherein the classifying comprises: designating a dispensing event by comparing an amount of the medication released in the dispensing event with a predefined priming dosage amount.

Embodiment 20 is the method of any of embodiments 13 through 19, wherein the classifying comprises: designating a dispensing event as a priming event upon determining that the dispensing event occurs immediately after a cartridge replacement of the medication injection device.

Embodiment 21 is the method of any of embodiments 13 through 20, wherein the classifying comprises: (1) designating a first dispensing event in the sequences as the therapeutic dose dispensing event upon determining that an amount of the medication released in a first dispensing event reaches a maximum limit of the medication injection device; (2) designating a second dispensing event immediately following the first dispensing event as the therapeutic dose dispensing event; and (3) accumulating the amount of the medication released in the first and second dispensing events for the calculation and tracking of a dosage amount for a patient.

Embodiment 22 is the method of any of embodiments 13 through 21, wherein the classifying comprises: (1) notifying a user when there is an ambiguity in the classifying of a dispensing event; and (2) receiving an input from the user to confirm whether the dispensing event is a therapeutic dose dispensing event or a priming event.

Embodiment 23 is an apparatus for differentiating a therapeutic dispensing event and a priming event in administering a medication, comprising: (1) a processor, and (2) a non-transitory memory including processor executable code, wherein the processor executable code upon execution by the processor configures the processor to: (a) determine, based on an input from a sensor of an injection device, whether the injection device is in contact with a patient or is held freely; (b) designate a dispensing event as a priming event when the device is held freely; and (c) designate a dispensing event as a dosing event when the device is in contact with a patient.

Embodiment 24 is the apparatus of embodiment 23, wherein the sensor of the injection device includes at least one of an accelerometer, a pressure sensor and a vibration sensor.

Embodiment 25 is the apparatus of embodiments 23 or 24, wherein the sensor of the injection device includes a capacitive sensor coupled to a needle of the injection device to detect a skin contact electrically.

Embodiment 26 is the apparatus of any of embodiments 23 through 25, wherein the sensor of the injection device includes a depth sensor or a capacitive sensor oriented toward an end of a needle of the injection device to detect a nearby surface.

Embodiment 27 is the apparatus of any of embodiments 23 through 26, wherein the processor executable code upon execution by the processor configures the processor to: (1) activate, prior to detecting a priming event, a locking mechanism on the injection device upon determining that the injection device is not oriented vertically upward; and (2) deactivate the locking mechanism after a priming event is detected.

Embodiment 28 is the apparatus of any of embodiments 23 through 27, wherein the injection device is configured to perform the first dispense of the medication as the priming event automatically.

### BRIEF DESCRIPTION OF THE DRAWINGS

FIG. 1 is a block diagram representing an example of an intelligent medicine administering system according to one embodiment of the disclosure;

FIG. 2 is a block diagram representing an example of a pen device forming part of the intelligent medicine administering system of FIG. 1;

FIG. 3 is a block diagram representing an example of the companion device of the intelligent medicine administering system of FIG. 1;

FIG. 4 is a block diagram representing an example of software architecture of data processing modules in accordance with some embodiments of the health management app of the intelligent medicine administrating system of FIG. 1;

FIG. 5 is a flowchart representation of a method for differentiating therapeutic and priming events in administering medication by a medication injection device in accordance with the present technology;

FIG. 6 is a flowchart representation of a method for differentiating a therapeutic dispensing event and a priming event in administering a medication; and

FIG. 7 is a flowchart representation of a method for determining the appropriate timing of the recommendation dismissal using prior priming and use patterns of the user according to one example embodiment.

### DETAILED DESCRIPTION

Disclosed herein are medicine-administering systems that among other features and benefits accurately provide health management capabilities for patients and caregivers. In one example aspect, a system includes a medicine injection device (also referred to as the “pen” or “pen device”), in communication with a patient's companion device (e.g., smartphone with the appropriate application software), in which the pen device is able to distinguish between a priming event or “prime” and a therapeutic dose or “dose” when medicine is dispensed from the injection device. The companion device includes a software application (“app”) having adaptive dose calculator and decision support modules to suggest the dose for the patient and provide control over several functionalities of the pen device.

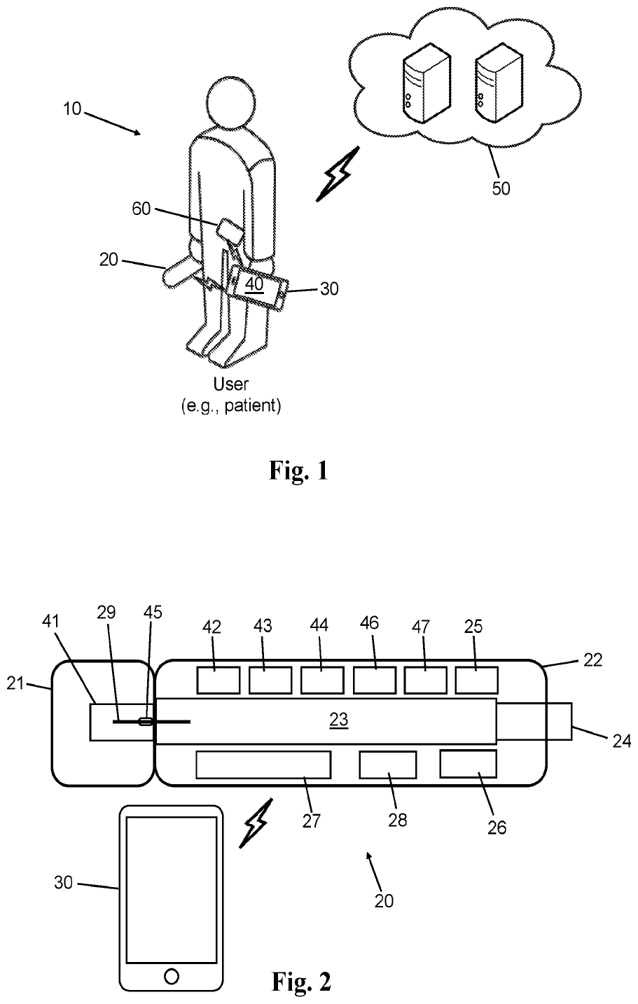

FIG. 1 shows a diagram of an example embodiment of an intelligent medicine administering system 10 in accordance with the present technology. The system 10 includes a pen device 20 in wireless communication with a mobile computing and communication device 30 of a patient user, also referred to as the user's companion device. The pen device 20 is operable to select, set and/or dispense a dose of the medicine for dispensing. In some implementations, the companion device 30 includes a smart phone, tablet, and/or wearable computing device, such as a smart watch, smart glasses, etc. In some implementations, the companion device 30 is in communication with other computing devices, such as a laptop and/or desktop computer, a smart television, or network-based server computer. The pen device 20 and/or companion device 30 includes a health management app 40 that may be associated with the pen device 20 and other smart health management accessories/devices of the intelligent medicine administering system 10.

In some embodiments, the system 10 includes a data processing system 50 in communication with the companion device 30 and/or the pen device 20. The data processing system 50 can include one or more computing devices in a computer system or communication network accessible via the Internet (also referred to as “the cloud”), e.g., including servers and/or databases in the cloud.

The health management app 40 is paired with the pen device 20, which may be a prescription-only medical device. In some implementations, the pairing (also referred to as bonding) of the companion device 30 to the pen device 20 indicates to the health management application 40 that the user is ready to use all features of the application, which can augment performance and provide important features to the intelligent medicine administering system 10. Thus in some implementations the act of pairing (bonding) therefore enables the full functionality of the health management app 40. For example, in some cases the pairing may enable the entire app 40, in which at least a portion of the health management app 40 may be disabled without the specialized pairing; whereas in other cases, the pairing may enable certain features of the health management app 40, which otherwise provides some limited features without the specialized pairing.

In some implementations, for example, the health management app 40 can monitor and/or control functionalities of the pen device 20 and provide a dose calculator and/or decision support modules that can calculate and recommend a dose of the medicine for the patient user to administer using the pen device 20.

The companion device 30 can be used to obtain, process and/or display contextual data that can be used to relate to the patient user's health condition, including the condition for which the pen device 20 is used to treat. In an illustrative example, the companion device 30 is operable to track the patient user's location; the patient user's physical activity including step count, movement distance and/or intensity, estimated calories burned, and/or activity duration; and/or the patient user's interaction pattern with the companion device 30. In some implementations, the app 40 can aggregate and process the contextual data to generate decision support outputs to guide and aid the patient user in using the pen device 20 and/or managing their behavior to promote better health outcomes in treating his/her health condition.

In some embodiments, for example, the system 10 can optionally include a sensor device 60 to monitor one or more health metrics of the patient user. Examples of health metric data monitored by the sensor device 60 include analytes, such as glucose, heart rate, blood pressure, user movement, or other. In some implementations, the sensor device 60 is a wearable sensor device such as a continuous glucose monitor (CGM) to obtain transcutaneous or blood glucose measurements that are processed to produce continuous glucose values. For example, the continuous glucose monitor can include a glucose processing module implemented on a stand-alone display device and/or implemented on the companion device 30, which processes, stores and displays the continuous glucose values for the patient user.

FIG. 2 shows a diagram of an example embodiment of the pen device 20 of the intelligent medicine administering system 10. The pen device 20 is structured to have a cap 21 configured to protect a medicine dispensing element (e.g. needle 29) and a body 22 configured to contain the medicine cartridge 23 (e.g., which can be replaceable). The pen device 20 is structured to include a dose dispensing mechanism 24 to dispense (e.g., deliver) the medicine contained in the medicine cartridge 23 out of the pen device 20; a dose setting mechanism 25 to select and/or set the dose to be dispensed; an operations monitoring mechanism 26 to determine that the pen device 20 is being operated and/or to monitor the operation of the dose being dispensed (e.g., such as a switch and/or sensor, or an encoder); and an electronics unit 27 that can include a processor, a memory, a battery or other power source, and a transmitter.

The pen device 20 is configured in communication with the patient user's mobile computing and communication device 30, e.g., such as the user's smart phone, tablet, and/or wearable computing device, such as a smart watch, smart glasses, etc. and/or a user's laptop and/or desktop computer, a smart television, or network-based server computer.

In some implementations of the system 10, for example, to use the pen device 20, the user first dials up a dose using a dose knob. The dose knob of the pen device 20 can be included as part of the dose setting mechanism 25 and/or the dose dispensing mechanism 24. For example, the dose may be adjusted up or down prior to administration of the dose. When the user applies a force against a dose dispensing button (e.g., presses against the dose dispensing button that is caused to protrude outward from the pen's body upon dialing the dose using the dose knob), a pushing component (e.g., also referred to as a ‘plunger’) of the dose dispensing mechanism 24 is depressed against an abutment of the medicine cartridge 23 loaded in the pen device 20 to cause the pen device 20 to begin to dispense the medicine, in which the quantity dispensed is in accordance with that set by the dose setting mechanism 25. In such implementations, the operations monitoring mechanism 22 of the pen device 20 will begin to sense movement of a rotating component or shaft that drives the plunger, for example, in which the movement is sensed through an encoder. In some examples, the encoder can be configured to sense the rotation of a component that is coupled to the drive shaft, and as the drive shaft rotates the plunger moves linearly; and therefore, by sensing rotation of the component, the movement of the drive shaft and the plunger is sensed. Movement of the encoder may be detected as data processed by a processor of the electronics unit 27 of the pen device 10, which can be used to measure the dose. In some implementations, the processor can then store the size of the dose along with a time stamp for that dose. In some implementations, the pen device 20 can then transmit the dose and related information to the companion device 30. In such implementations when the dose is transmitted, the data associated with the particular transmitted dose is marked in the memory of the pen device 20 as transmitted. In such implementations if the dose was not yet transmitted to the companion device 30, then the data associated with the dose will be transmitted at the next time a successful communication link between the pen device 10 and the companion device 30 is established.

The operations monitoring mechanism 22 of the pen device 20 can include a sensor that can utilize any method of sensing rotary or linear movement. Non-limiting examples of such sensors include rotary and linear encoders, Hall effect and other magnetic based sensors, linearly variable displacement transducers, or any other appropriate method of sensing known in the art.

The dose dispensing mechanism 24 of the pen device 20 can include a manually powered mechanism or a motorized mechanism. In either case, a force (e.g., either produced by the patient or by an electrically-powered motor) pushes on the plunger of the dose dispensing mechanism 24 to in turn force a receiving plunger of the medicament vial or cartridge 23 to deliver the specific amount of the medicament. In some implementations, for example, the dose dispensing mechanism 24 can be adjusted to deliver the dose over a different period of time. In one example, the dose dispensing mechanism 24 can be operated such that the plunger is pushed in by an adjustable tension spring or change the speed of the motor to inject the dose over a time frame (e.g., 1 s, 5 s or other) to aid in reducing the pain of dosing. In one example, the dose dispensing mechanism 24 can be operated over a much longer period of time, e.g., to better match the dynamics of carbohydrates, which can be like an extended bolus with a pump.

The health management app 40 of the companion device 30 associated with the pen device 20 provides a user interface to allow the user to manage his/her health-related data. In some implementations, for example, the health management app 40 can be configured to control some functionalities of the pen device 20. In some implementations, for example, the health management app 40 provides an interactive user interface to allow a user to manage settings of the pen device 20, and settings for the companion device 30 (e.g., smart phone, tablet, or wearable computing device) that can affect the functionality of the system 10. In implementations, for example, the companion device 30 is an independent portable device that the user may carry on his/her person. In example embodiments of the independent portable companion device 30, the companion device 30 includes a data processing unit, wireless communication unit to allow the device to communicate with the pen device 20, and a display unit.

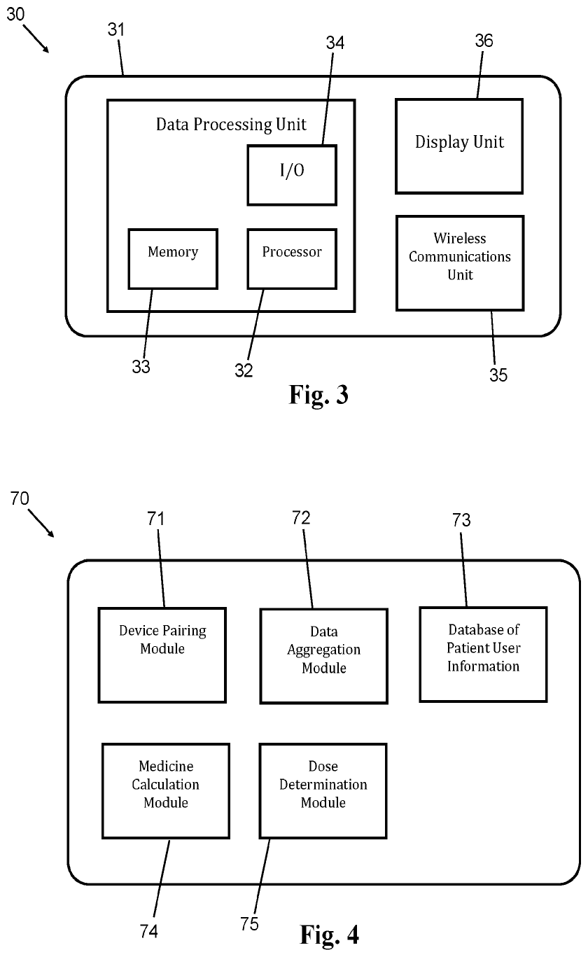

FIG. 3 shows a block diagram of an example embodiment of the companion device 30 (e.g. or “companion computing device”, or “computer”) of the intelligent medicine administering system 10. The data processing unit 31 of the companion device 30 includes a processor 32 to process data, a memory unit 33 in communication with the processor 32 to store data, and an input/output unit (I/O) 34 to interface the processor 32 and/or memory 33 to other modules, units or devices of the companion device 30 or external devices. For example, the processor 32 can include a central processing unit (CPU) or a microcontroller unit (MCU). For example, the memory 33 can include and store processor-executable code, which when executed by the processor 32, configures the data processing unit 31 to perform various operations, e.g., such as receiving information, commands, and/or data, processing information and data, and transmitting or providing information/data to another device. In some implementations, the data processing unit can transmit raw or processed data to a computer system or communication network accessible via the Internet (referred to as ‘the cloud’) that includes one or more remote computational processing devices (e.g., servers in the cloud). To support various functions of the data processing unit, the memory 33 can store information and data, such as instructions, software, values, images, and other data processed or referenced by the processor 32. For example, various types of Random Access Memory (RAM) devices, Read Only Memory (ROM) devices, Flash Memory devices, and other suitable storage media can be used to implement storage functions of the memory unit 33. The I/O 34 of the data processing unit 31 can interface the data processing unit 31 with the wireless communications unit 35 to utilize various types of wired or wireless interfaces compatible with typical data communication standards, for example, which can be used in communications of the data processing unit 31 with other devices such as the pen device 20, via a wireless transmitter/receiver (Tx/Rx) unit, e.g., including, but not limited to, Bluetooth, Bluetooth low energy, Zigbee, IEEE 802.11, Wireless Local Area Network (WLAN), Wireless Personal Area Network (WPAN), Wireless Wide Area Network (WWAN), WiMAX, IEEE 802.16 (Worldwide Interoper-ability for Microwave Access (WiMAX)), 3G/4G/LTE cellular communication methods, NFC (Near Field Communication), and parallel interfaces. The I/O 34 of the data processing unit 31 can also interface with other external interfaces, sources of data storage, and/or visual or audio display devices, etc. to retrieve and transfer data and information that can be processed by the processor 32, stored in the memory unit 33, or exhibited on an output unit of the companion device 30 or an external device. For example, a display unit 36 of the companion device 30 can be configured to be in data communication with the data processing unit 31, e.g., via the I/O 34, to provide a visual display, an audio display, and/or other sensory display that produces the user interface of the health management application 40. In some examples, the display unit 36 can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc.

By way of example, when a dosing event occurs (e.g., an amount of fluid is dispensed from the pen device 20), a time stamp associated with the dosing event is recorded by the processing unit of the pen device 20 (e.g., stored in the memory of the pen device 20). For example, the time stamp may be the current time or a time where a count-up timer is used. When the dose information is eventually transmitted to the health management app 40 of the companion device 30, the time stamp and/or a ‘time-since-dose’ parameter is transmitted by the pen device 20 and received by the companion device 30 and stored in the memory 33 of the data processing unit 31 of the companion device 30. In some implementations, for example, the time of the dose can be determined without the pen device 20 having to know the current time. This can simplify operation and setup of the pen device 20. In some implementations, for example, a user time is initialized on the pen device 20 from the companion device 30, in which the user time is used for dose time tracking. Using the system 10, the health management app 40 can know the time of the dose relative to the current time.

Once the companion device 30 receives the dose related information (e.g. which can include the time information and dose setting and/or dispensing information, and other information about the pen device 20 related to the dosing event), the companion device 30 stores the dose related information in memory 33, e.g., which can include among a list of doses or dosing events. In some implementations, for example, via the user interface of the health management app 40, the companion device 30 allows the patient to browse a list of previous doses, to view an estimate of current medicament active in the patient's body (“medicament on board”) based on calculations performed by a medicine calculation module 74 of the health management app 40, and/or to utilize a dose determination module 75 of the software application to assist the patient regarding dose setting information on the size of the next dose to be delivered. For example, the patient may enter carbohydrates to be eaten and current blood sugar, and the companion device 30 would already know insulin on board. Using these parameters, a suggested medicine dose (e.g., such as insulin dose), calculated by the dose determination module 75, may be determined. In some implementations, for example, the companion device 30 can also allow the patient to manually enter boluses into the pen device 20 or another medicine delivery device. This would be useful if the patient was forced to use a syringe, or if the battery in the pen device 20 was depleted.

Example embodiments and implementations of the disclosed intelligent medicine administering system 10, including the health management app 40 operable on a companion device 30 able to communicate with a medical device (e.g., medicine dispensing device such as a pen device 20), are described. Some examples of features of an intelligent medicine administering system that can be used with the example methods, devices and systems for providing a prescription-regulated software controls on the system are described in commonly-owned U.S. Pat. No. 9,672,328 B2, entitled “Medicine Administering System Including Injection Pen and Companion Device”.

While the disclosed embodiments described herein are primarily descried using as examples, diabetes management systems and methods involving an insulin pen, health management app associated with the insulin pen, and/or glucose monitoring devices to facilitate understanding of the underlying concepts, it is understood that the disclosed embodiments can also include treatment of other health conditions using other medications by the pen device, health management app, and/or monitoring of other analytes by sensor devices. For example, the disclosed prime differentiation techniques can be used in conjunction with other dispensing devices and medications other than insulin that require priming.

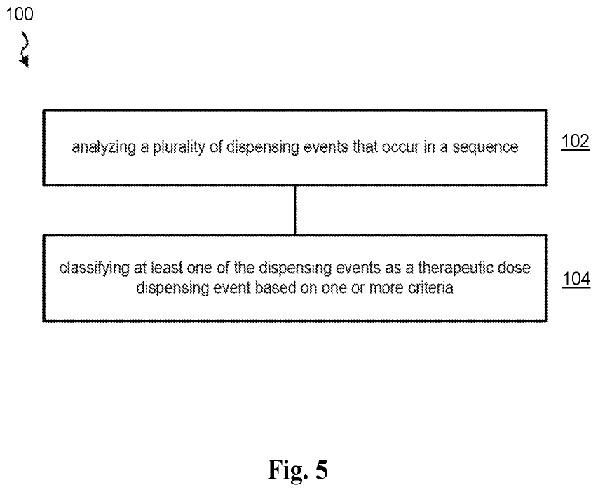

FIG. 4 shows a block diagram of a software architecture 70 of data processing modules in accordance with some example embodiments of the health management app 40 of the intelligent medicine administrating system 10. In some embodiments, some or all of the data processing modules of the software architecture 70 of the health management app 40 are resident on the companion device 30. In some implementations of the example software architecture 70, the modules operate to control certain functionalities of the companion device 30, which includes aggregating and processing output signals transmitted by the medical injection device (e.g., pen device 20) to execute a device pairing for validation of the medicine injection device and unlocking of some or all functionality of the health management app 40 on the companion device 30.

In some embodiments of the software architecture 70, some or all of the data processing modules are resident on the companion device 30, e.g., resident in the data processing unit 31. In some embodiments of the software architecture 70 of the system 10, some of the data processing modules can be resident on the pen device 20, e.g., resident in the electronics unit 27. In some embodiments of the software architecture 70 of the system 10, some of the data processing modules may be resident on smart accessories configured for use with standard (non-intelligent) dispensing devices. Similarly, in some embodiments, for example, some of the data processing modules of the software architecture 70 can be embodied as part of a data processing system 50 in the cloud (e.g., resident on one or more cloud computers).

The data processing modules of the health management app 40 on the companion device 30 may include different or the same data processing modules of the software architecture 70 resident on the pen device 20. For example, one of the data processing modules that can be resident on and implemented by the pen device 20 includes a device pairing module 71 to receive and process the output signals from the pen device 20 to pair with the health management app 40 resident on the companion device 30.

In some implementations, the pen device 20 can be paired to the companion device 30 to enable some or all protected features of the health management app 40 associated with a specific user of the pen device 20 and/or data management on the companion device 30 which require secure access for use by the patient user. The secure pairing methods ensure that the health management app 40 resident on the companion device 30 is specifically associated with a particular pen device 20 belonging to the patient user, e.g., who may have a prescription corresponding to use of some or all features of the health management app 40.

In some embodiments of the software architecture 70 for the health management app 40, the data processing modules may also be configured to process health metric data and contextual data obtained by the health management app 40 on the companion device 30, from the pen device 20, from the sensor device 60, and/or from other devices of the patient user and/or apps on the companion device 30. In some embodiments, for example, the software architecture 70 includes a data aggregation module 72 to obtain the health metric data from a device, such as the pen device 20, a sensor device 60, and/or other devices or apps in communication with the companion device 30. The software architecture 70 includes or is in communication with a database 73 to store the health data and contextual data associated with the patient user and populations of patient users. In various embodiments, the database 73 can be resident on the data processing system 60, e.g., in the cloud. In some embodiments, the software architecture 70 includes a medicine calculation module 74 to estimate the current medicament active in the patient's body (“medicament on board” or “insulin on board” or IOB). In some embodiments, the software architecture 70 includes a dose determination module 75, such as a dose calculator module, to autonomously calculate a dose of the medicine associated with dose injections from the pen device 20 based on time-relevant and context or circumstances-relevant data specific to the patient user of the pen device 20. The example modules shown in the software architecture diagram can be organized to provide data to one another in various ways, including directly (e.g., module-to-module) and/or indirectly (e.g., via an intermediary module), based on a periodic, an intermittent, and/or a per-request basis.

The embodiments of the disclosed technology, among other features and benefits, provide multiple methods for a pen device or smart injector 20 and/or health management app 40 to differentiate between doses and primes. In some scenarios, a method for evaluating or detecting medication doses versus primes may be ambiguous, and in these scenarios, it can be useful for a datalogger component to record or otherwise retain the information regarding the ambiguity, so it may prompt the user for manual confirmation. Typically, overdosing on insulin is more harmful than taking too little, so in the event of an ambiguity it is safer to assume that a dispense event was taken as a dose. For example, in subsequent calculations, the presence of an ambiguity may be taken into account to artificially decrease doses the user will take. In contrast, mistaking a dose as a prime would cause the system to under-estimate the amount of insulin a user has taken, causing subsequent calculations to recommend too much insulin to be dispensed.

In the description that follows, subheadings are used to provide a proper flow for understanding of the disclosed embodiments. It should be understood, however, that the various features of the disclosed embodiments are described throughout this patent document and their description is not limited to what is provided under a specific subheading.

Dispensing Pattern Recognition

In operating a medicament dispensing device, such as pen device 20, a user typically primes the dispensing device 20, possibly multiple times, before applying a therapeutic dose. Accordingly, by way of example, the intelligent medicine dispensing system 10 may evaluate a pattern or sequence of dispense events within a given time window, designating the last event in the pattern as a therapeutic dose and designating events prior to the last event in the pattern as priming events. However, there may be several scenarios that occur during normal use of the intelligent medicine dispensing system 10 for which the last event in a sequence of dispensing events is not actually the therapeutic dose, including scenarios in which one or more therapeutic dose events occur prior to the last event in a sequence of dispensing events. The intelligent medicine dispensing system 10 is configured to recognize and account for these scenarios when designating a dispensing event as prime or dose. By way of example, scenarios which may require alternative designation analysis may include (but are not limited to) the following example scenarios:

1. If the dispensed amount is above a predefined threshold, the intelligent medicine administering system 10 may designate the dispense event as a therapeutic dose even if it was not the last dispense event in a sequence. This is because dosage amounts in priming events are typically small (e.g., 2 units or less). A large injection indicates that the dispensed amount was likely a therapeutic dose.

2. Typically, users need to prime a new cartridge to enable the dispensing of insulin. The intelligent medicine administering system 10 may thus designate the first dispense event after a cartridge replacement as a priming event.

3. If a dose calculator is used after an initial dispense event, indicating that the user has not yet applied a therapeutic dose, the intelligent medicine administering system 10 may designate dispense events taken up to that point as priming events and may reset the dose/prime window time.

4. Any dose for which the volume of injected medicine is at the maximum capacity of the pen device 20 (commonly 30 u, 60 u, or 80 u) may be designated as a therapeutic dose, and the amount injected may be accumulated towards the total therapeutic dose. For example, when a user requires a dose amount that exceeds the capacity of their pen device 20 (e.g., 90 u), the device's maximum dose is first administered (e.g., 80 u), followed by the additional amount needed (e.g., 10 u). In such a situation, the intelligent medicine administering system 10 may designate both dispense events as therapeutic doses.

5. If the administration of a dose exhausts the supply of medicine remaining in the cartridge 23 of the pen device 20 and is followed by a cartridge replacement within the dose time window, the final dispense event before the occurrence of the cartridge replacement may be designated as a therapeutic dose even if more primes and/or doses follow within the time window.

6. If two dispense events are the same size (e.g., 2 u followed by another 2 u), the intelligent medicine administering system 10 may prompt the user to confirm that they were therapeutic doses and/or priming events. The system 10 may also prompt the user to confirm that the occurrence was not an accidental double-dose.

7. If two retraction events (e.g. backward movement of the plunger) occur within a defined time threshold (e.g. 10 minutes), then the intelligent medicine administering system 10 may designate any dispensing event occurring between the retraction events as non-therapeutic (e.g. prime and/or null).

8. In the context of an initial use of a new device, if one or more dispense events occur followed by a retraction event, then the system 10 may designate the dispense events as non-therapeutic (e.g. prime, training, and/or null).

In some embodiments, a manually-actuated injector pen device 20 that senses advancement of the cartridge plunger (or detects mechanism movement related to plunger advancement, such as knob or screw rotation) may differentiate between pauses in a user's movement during a single injection event and/or separate injection events. The system 10 may use a preset timeout threshold, such as one second, so that movements within the preset timeout threshold are considered to be a single dispense event, and movements outside of the timeout threshold are considered separate dispense events (e.g., a prime and a dose).

The challenge with a fixed timeout threshold is that users dispense at different rates and with different pauses between dispense events. For example, some users may dial and dispense very quickly, resulting in a very short timeout threshold to differentiate two separate dispense events, while other users may dispense very slowly, resulting in longer pauses between movements within a single injection. A longer timeout threshold is needed for such users to avoid interpreting the single injection as multiple smaller injections. To account for this disparity, the intelligent medicine administering system 10 may be configured to determine a user-specific timeout threshold using one or more of the following methods, presented by way of example:

1. The intelligent medicine administering system 10 may be configured to enable the user to manually select a desired timeout threshold based on the user's behavior.

2. The pen device 20 may be configured to signal to the user (e.g., via an integrated light or sound device, or by triggering an immediate notification in the user's companion device 30) when a dispense event is considered completed. If the user notices this notification during an injection, they may then manually correct the record of that injection event by interacting with the companion device 30, and if needed adjust the timeout threshold settings to avoid future alerts.

3. The intelligent medicine administering system 10 may analyze the user's history of dose timing (e.g., from detected injection events, including past manual corrections or overrides) and adjust the timeout threshold accordingly. For example, the system 10 may apply a fixed percentage to the user's average time between dose and prime to determine the time timeout threshold.

4. In some instances, a user dispensing very slowly may be prone to pause momentarily partway through the dispensing event, or may be moving so slowly that the pen device 20 may not sense movement for an extended period of time. To account for such instances, the intelligent medicine administering system 10 may be configured to detect the speed of user movement (e.g. revolutions per minute (RPM) of a rotating screw or linear velocity of the plunger advancing) and use this detected speed to adjust the user-specific timeout threshold. For example, the system 10 may increase the timeout threshold when the user injects very slowly and decrease the timeout threshold when the user injects more quickly.

5. In some embodiments, the intelligent medicine administering system 10 may be configured to define a minimum timeout threshold and a maximum timeout threshold. For example, the system 10 may designate multiple movements within the minimum timeout threshold as a single movement, and designate multiple movements beyond the maximum timeout threshold as multiple movements. For movements that fall between the minimum and maximum timeout thresholds, the system 10 may prompt the user for confirmation (e.g., by presenting an interactive prompt on a user interface of the companion device 30 or pen device 20) and/or analyze additional pattern recognition methods to make a determination.

User Adaptation

While priming behavior may vary from user to user, any single user often exhibits consistent priming behavior. Thus, analysis of a user's specific dispensing history may inform the determination of whether a questionable dispensing event is a prime or a dose event. By way of example, the intelligent medicine administering system 10 of the present disclosure may be configured to monitor and analyze a specific user's dispensing activity history to identify (1) the user's typical dispense amounts in prime and dose event, and (2) the user's typical priming behavior. This data, by itself or in combination with other methods disclosed herein, may help inform the prime/dose determination.

For example, in identifying the user's typical dispense amounts for therapeutic dose and priming events, the system 10 may determine dispense amounts such as the average, maximum, minimum, upper 75th percentile, and/or lower 75th percentile. In some implementations, for example, the intelligent medicine administering system 10 may designate a dispense event larger than the user's maximum past priming amount as a therapeutic dose. In some embodiments, the system 10 may designate a dispense event larger than the user's past lower 75th percentile dose amount as a therapeutic dose.

By way of example, in identifying the user's typical priming behavior, the system 10 may analyze the user's dispense event history to determine the frequency of prime events. For example, if the system 10 determines that less than a predetermined percentage (e.g. 10%) of the user's recorded therapeutic doses are accompanied by a prime event, then the system 10 may record all dispense events as therapeutic doses. Similarly, if the system 10 determines that greater than a predetermined percentage (e.g. 90%) of the user's therapeutic doses are accompanied by a prime event, then the system may record the first dispense event in a sequence as a priming event.

In some embodiments, for example if the user uses a dose calculator (e.g. dose determination module 75 of the health maintenance app 40) or is presented with calculated doses to determine a dosage amount, or the system 10 determines the user's glucose value and Insulin on Board (IOB) to calculate a proper correction dosage amount for the user (e.g. by way of the medicine calculation module 74), then the intelligent medicine administering system 10 may use the dosage amount to define the dispense threshold that differentiates therapeutic doses from the priming events. For example, if the smallest therapeutic dose is significantly above a typical priming amount (e.g. 2 units), then the system 10 may designate all small dispense events (e.g. 2 units or less) as priming events. However, if the smallest therapeutic dose calculated is comparable to or less than a typical prime size, then the system 10 may designate a small dispense event as a therapeutic dose unless other data available to and analyzed by the system 10 indicates that the dispense event is a priming event.

In some instances, a dispense event may be inadvertent. For example, a user may inadvertently dispense a small amount of medicine on the floor during handling, etc. Logging these inadvertent movements as therapeutic doses may create inaccurate records and lead to inaccurate calculations in the future. Thus, the intelligent medicine administering system 10 may disregard such a dispense event, record the event as a null event, or prompt the user to confirm whether the dispense event is valid (e.g. dose or prime) by analyzing the user's dose history data and determining that one or more of the following example conditions are satisfied: (1) the amount of medicine dispensed is smaller than the user's past therapeutic doses and/or priming events; (2) the dispensing occurs outside the user's normal dose reminder windows; (3) the dispensing event occurs outside the typical time windows of past dosing events; and/or (4) there is a single dispense event for a user whose past data indicates that they usually prime before applying a therapeutic dose (and therefore typically have two or more dispense events in a row).

In some embodiments, the intelligent medicine administering system 10 may be configured to use dose calculator parameters to set a threshold value for an unrealistic dose amount for the user, to help determine whether a dispense event is valid. By way of example, the threshold value may be based upon a preset maximum dose setting, an offset of the preset maximum dose setting, or a multiple of the preset maximum dose setting. In some embodiments, the system 10 may set the threshold value based upon a dose calculation of a large correction with a large meal, using the set dose calculator parameters.

Manual Setting

As discussed above, in some embodiments, manual input from the user may provide accurate information and can help detect infrequent errors even when automatic dose/prime detection is available. By way of example, the intelligent medicine administering system 10 may also prompt the user to manually input certain data initially until enough use data is recorded in the system's database 73 to enable the system 10 to automatically recognize and/or anticipate the user's typical behavior. In some embodiments, this user interaction may occur by way of graphic user interface on a display unit of the user's companion device 30 or pen device 20. In some embodiments, other means of interaction such as voice or audio prompts and voice or audio commands may be used.

In some implementations, the intelligent medicine administering system 10 may display the dose amount detected by the pen device 20 in real-time. The dose information can be displayed on the pen device 20 or on display unit 36 of a user's companion device 30 (e.g. smart phone or other smart device), allowing the user to verify that their actions were correctly interpreted by the system 10. The system 10 may provide the user with an opportunity to correct the data logs if necessary. In some embodiments, the system 10 may prompt the user for confirmation of a dose amount or whether a dispense event was a therapeutic dose or a priming event (or a null event) if there is any ambiguity. For example, if the system 10 detects a dispense event immediately before the user uses a dose calculator, the system 10 may prompt the user to confirm whether this was a therapeutic dose or a priming event (or a null event) before enabling access to the dose calculator 75, ensuring that the system 10 has an accurate determination of insulin in the user's body before calculating the imminent dose.

Implicit Detection and Forced Priming

By way of example, in some embodiments, the pen device 20 may include an accelerometer 28 configured to sense the difference between a pen device 20 held freely in the air and a pen device 20 held steady against a user's skin. For example, when the accelerometer 28 indicates large movements and/or movements at a lower frequency during the dispense event (or immediately prior to the completion of the dispense), the intelligent medicine administration system 10 may determine that such movements are due to the pen device 20 being held freely in the air and therefore the system 10 may designate the dispense event as a priming event. When the accelerometer 28 indicates small movements and/or movements at a higher frequency during the dispense event, the system 10 may determine that such movements are due to the device being held steadily against the user's skin and therefore the system 10 may designate the dispense event as a therapeutic dose.

In some embodiments, the system 10 may require that the user primes the pen device 20 prior to enabling use of the dose calculator 75 (or prior to displaying a calculated result on the display unit 36 of the companion device 30). In such embodiments, the system 10 may consider dispense events occurring before dose calculator 75 usage to be priming events and dispense events occurring after the dose calculator 75 usage to be therapeutic doses. To minimize waste of insulin that results from priming a pen device 20, the system 10 may require priming only when the system 10 determines that a therapeutic dose is recommended, or a therapeutic dose above a certain threshold amount is needed.

In some embodiments, the pen device 20 may be configured to force priming by mechanically locking the dispense mechanism 24 unless and until a dispense event occurs while the distal or dispensing end of the pen device 20 is pointed upward. For example, the accelerometer 27 or other attitude indicator may sense the orientation of the pen device 20 and relay that information to the processing unit of the pen device 20 or the user's companion device 30. In some implementations, a mechanical weight can be used to unlock the pen device 20 for priming when held vertically with the distal or dispensing end pointed upward. Since it is difficult for the user to administer a dose with the pen device 20 pointing upward, locking the pen device 20 effectively forces the user to properly prime the pen device 20. The lock mechanism may apply to the dispensing mechanism 24 (e.g. an injection button) or to a shroud 41 or standoff that prevents the user from injecting the needle 29 while still allowing the user access to install a needle, remove a needle cover, or prime liquid out of the needle.

In some embodiments, the intelligent medicine administering system 10 may automatically prime the pen device 20 so that the user does not need to dispense a separate priming event. In such cases, the system 10 may cause the plunger to automatically advance a small amount as the priming event when the dose selection mechanism 25 is actuated (for example). In such devices, the system 10 records all user dispense events as therapeutic doses.

Explicit Detection

In some embodiments, the intelligent medicine administering system 10 may be configured to detect whether the pen device 20 is near to or in contact with skin to determine if a dispense event is a therapeutic dose or a prime event. For example, the pen device 20 may be configured to detect a physical contact of the injector with the user. The pen device 20 may also be configured to perform electrical detection of the needle 29 being embedded in tissue, or detection of the injector's proximity to the user.

In some implementations, the pen device 20 may have a shroud 41 around the needle 29 that allows liquid to exit for priming but guards against accidental contact of the needle 29 with the user or with other objects in the environment such as a table or the ground. When the distal end of the pen device 20 is pressed against the user's skin, the shroud 41 translates back to allow the needle 29 to penetrate the user's skin. By way of example, the pen device 20 may include a switch configured to detect translation of the shroud 41, which indicates that the dispense event is a therapeutic dose. Thus, when the switch detects translation of the shroud 41 prior to a dispense event, the intelligent medicine administering system 10 records the dispense event as a therapeutic dose. When the switch fails to detect the shroud's translation during the dispense event, the system 10 records the dispense event as a priming event.

In some embodiments, a pressure sensor 42 or vibration sensor 43 provided on the needle or needle attachment may detect forces and vibration exerted by the user's skin on the needle. By way of example, in response to the presence of force or vibration in the needle as detected by the pressure or vibration sensor, the system 10 may record an associated dispensing event as a therapeutic dose. In the absence of a detection of force or vibration in the needle by the pressure or vibration sensor, the system 10 may record the dispensing event as a priming event, absent further data that would indicate otherwise.

In some embodiments, a capacitive sensor 44 may be electrically coupled to the needle 29 to electrically detect skin contact. The pen device 20 may have an electrically conductive cartridge septum, or a conductive needle hub configured to make electrical contact with the disposable needle 29.

In some embodiments, a coil 45 may be positioned around the needle to measure its inductance, which changes once the needle 29 makes contact with skin. The system 10 can then designate dispense events as therapeutic doses or priming events based on the measured needle inductance. In some cases, the measured inductance may be slightly affected by the amount of insulin remaining in the cartridge 23. Therefore, during priming when the needle 29 is not contacting the user, the system 10 may use the inductance to approximate the amount of drug remaining in the cartridge 23. Additionally, the system may use this measured inductance to detect whether a full or partly-empty cartridge is installed after a cartridge change.

In some embodiments, two or more electrical contacts positioned at the needle end of the pen device 20 (one of which may be the needle 29 itself) may sense when an electrical circuit is completed (closed), indicating that the contacts are touching the user's skin and the system 10 may designate the dispense event as a therapeutic dose.

In some configurations, a new medicine cartridge 23 installed into a pen device 20 requires some movement of the pen device 20 to adjust the drive mechanism against the cartridge's plunger before medicine can be dispensed. In these configurations, if the pen device 20 detects negligible force against the drive mechanism, then the system 10 may determine that the drive mechanism has not yet contacted the cartridge plunger and as a result may designate the movement as a priming event in accordance with some embodiments.

In some implementations, the pen device 20 may have a thermal sensor 46 at the needle end, configured to detect body heat of a user. By way of example, the system 10 may use information received from the thermal sensor to determine whether the pen device 20 is being held against a user or held freely, and thus whether a concurrent dispense event is a dose or a prime event.

In some embodiments, a depth sensor 47 (e.g. a laser, optical, or ultrasonic) may be oriented toward the needle end of the pen device 20. When the depth sensor detects a surface nearby (e.g., within the length of a typical pen needle, such as 5 mm) this indicates that the needle 29 is injected into the surface and the system 10 may identify the dispense event as a therapeutic dose. If the depth sensor cannot detect a nearby surface, or it detects a distance greater than a pen needle, then the system 10 may designate the dispense event as a priming event.

By way of example, the intelligent medicine administrating system 10 and/or pen device 20 may use a single method or multiple methods disclosed herein in combination to differentiate a therapeutic dose from a priming event. For example, the system 10 may utilize pattern recognition, a depth sensor, and an accelerometer. In some implementations, the system 10 may aggregate different inputs probabilistically, such as with a voting scheme, so that the majority of multiple inputs are accepted as truth. In some embodiments, the system 10 may conservatively assume that any of the inputs indicating a therapeutic dose is correct. In this mode, the system may only identify a dispense event as a priming event if all inputs indicate it as such. In some implementations, if all inputs do not agree and corroborate the dose/prime designation, the user is prompted by the system 10 to provide confirmation. In some embodiments, the system 10 may use a primary method to determine therapeutic/priming event designation. When the result of the primary method is ambiguous, the system 10 may use a secondary method to assist the determination.

By way of example, FIG. 5 is a flowchart representation of a method 100 for differentiating therapeutic and priming events in administering medication by a medication injection device in accordance with the present technology. The method 100 includes, at operation 102, analyzing a plurality of dispensing events that occur in a sequence. The method 100 includes, at operation 104, classifying at least one of the dispensing events as a therapeutic dose dispensing event based on one or more criteria.

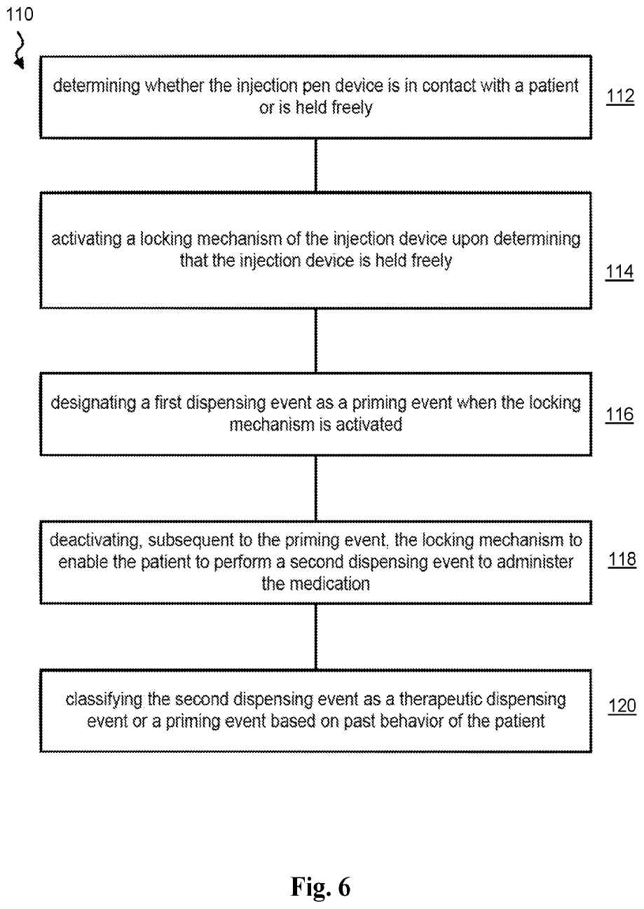

By way of example, FIG. 6 is a flowchart representation of a method 110 for differentiating a therapeutic dispensing event and a priming event in administering a medication. The method 110 includes, at operation 112, determining, based on an input from a sensor of an injection device, whether the injection device is in contact with a patient or is held freely. The method 110 includes, at operation 114, activating a locking mechanism of the injection device upon determining that the injection device is held freely. The method 110 includes, at operation 116, designating a first dispensing event as a priming event when the locking mechanism is activated. The method 110 includes, at operation 118, deactivating, subsequent to the priming event, the locking mechanism to enable the patient to perform a second dispensing event to administer the medication. The method 110 also includes, at operation 120, classifying the second dispensing event as a therapeutic dispensing event or a priming event based on past behavior of the patient.

Prospective Prime Differentiation & Recommendation Dismissal

As previously mentioned, in some instances users may use a dose calculator 75 to help them ensure they are injecting the correct therapeutic dose. By way of example, a dose calculator 75 is a tool for people with diabetes to calculate a rapid-acting insulin dose, based upon planned carbohydrate intake, current glucose level and insulin still active from prior injections. The output of a dose calculator 75 is a dose recommendation displayed by the intelligent medicine administering system 10 on the user interface 36 of the companion device 30 and/or pen device 20. For a good experience, the dose recommendation should be displayed until the user injects that dose, and then the system 10 may update the user interface of the companion device 30 to remove the recommendation and optionally show the dose has been taken, giving positive confirmation of the action. Moreover, if a user is administering a multi-part dose, for example dosing 50 U total in 2 parts—30 U then another 20 U injection, the recommendation may be updated in real time to inform the user how much of the dose recommendation is left to inject on the next injection.

Various methods are described herein by which the intelligent medicine administering system 10 (e.g. the pen device or smart injection pen 20, companion device 30 and/or health management app 40) may prospectively distinguish between a prime dose and a therapeutic dose and as a result remove the dose recommendation from the user interface of the companion device 30 (also referred to herein as “recommendation dismissal”). In some embodiments, the system 10 may incorporate learned prior priming and use patterns, specific to a user and customized to the user's own recent behavior, to determine whether a dose was therapeutic to inform the appropriate timing of the recommendation dismissal. In some embodiments, the system 10 may use confidence scoring to determine whether a dose was therapeutic. In some embodiments, the system 10 may incorporate user interactions to improve the user experience. In some embodiments, the system 10 may update the dose recommendation in real time to inform users administering therapeutic doses over several injections.

Learned Prior Priming and Use Patterns

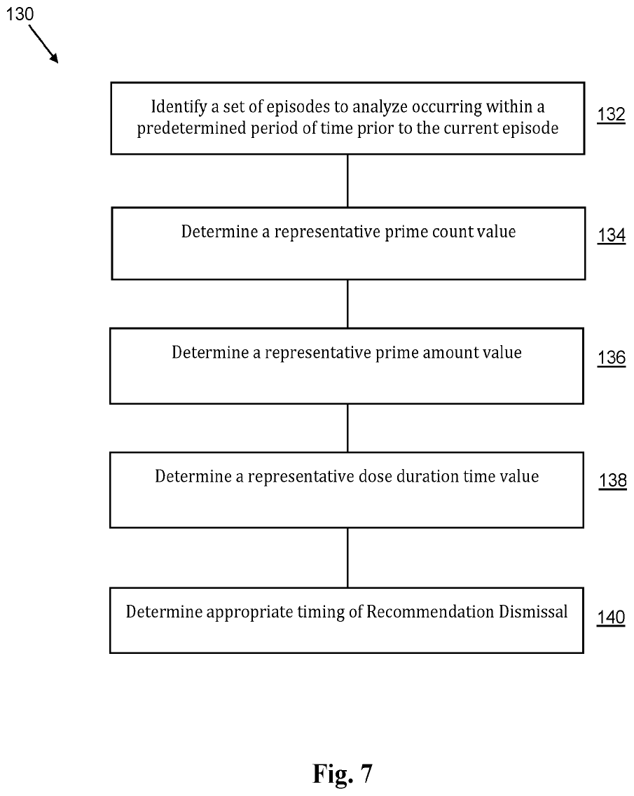

FIG. 7 is a flowchart representation of a method 130 for determining the appropriate timing of the recommendation dismissal using prior priming and use patterns of the user according to one example embodiment. For the purposes of this disclosure, the “insulin recommendation” is the last dose amount recommended by the system dose calculator 75 before the user begins dispensing insulin. An “episode” is the collection of dose activity (e.g., prime and/or therapeutic) within a predetermined period of time (e.g., 10 minutes) following the insulin recommendation. The system 10 determines the episode start time by referencing the stored datetime value of the insulin recommendation.

By way of example, the system 10 may determine the user's rolling prior prime behavior by analyzing the user's prime activity from within a set of episodes starting within a predetermined period of time prior to the start time of the current episode. The predetermined period of time may be any period of time that is sufficient to establish a pattern of behavior by the user. By way of example, the predetermined time period may be one week, resulting in the system determining a rolling week prime behavior. Once the set of episodes 132 has been determined, the system 10 uses stored data from those episodes to calculate a representative or typical prime count value 134, a representative or typical prime amount value 136, and a representative dose duration value 138, all specific to the user, that the system 10 will later use to determine the appropriate timing of recommendation dismissal.

In some implementations, the system 10 may determine the representative or typical prime count value 134 using any of a variety of methods, including but not limited to finding a median prime count value based on the user's recorded recent behavior, finding a mean prime count value based on the user's recorded recent behavior, prompting the user to select a prime count to be used, finding a mean prime count value across a population of a subset thereof, relying on a default prime count value preselected in the software app 40, and the like. In the instant example, the system 10 determines the representative or typical prime count value 134 by finding the user's median prime count value in a rolling week of prime behavior. By way of example, to establish the median prime count value, the system 10 calculates the count of prime occurrences per episode within the rolling week (for example) subset of prior episodes. For any episodes in the subset that do not have prime occurrences, the system 10 assigns a prime count value of zero to that episode. For episodes that do have prime occurrences, the system 10 counts how many occurrences were labeled as prime at the time of occurrence, and assigns that prime count to that episode. The system 10 then finds the median of the prime count values, arranged numerically from least to greatest.

In some implementations, the system 10 may determine the representative or typical prime amount value 136 using any of a variety of methods, including but not limited to finding a median prime amount value based on the user's recorded recent behavior, finding a mean prime amount value based on the user's recorded recent behavior, prompting the user to select a prime amount to be used, finding a mean prime amount value across a population of a subset thereof, relying on a default prime amount value preselected in the software app 40, and the like. In the instant example, the system 10 determines the representative or typical prime amount value 136 by finding the user's median prime amount value in a rolling week of prime behavior. By way of example, to establish the median prime amount value 136, the system 10 retrieves stored dose amount data for doses labeled as “Prime” according to “type” within the rolling week (for example) subset of prior episodes, and excludes any statistical outlier events, for example including but not limited to cartridge change events. For any episodes in the subset that do not have prime occurrences, the system 10 assigns a prime amount value of zero to that episode. The system 10 then finds the median of the prime amount values, arranged numerically from least to greatest.

If there are less than a predetermined number of episodes in the predetermined time period, then the system 10 may access stored data for episodes occurring outside of (e.g. earlier than) the predetermined time period in order to include the predetermined number of episodes in the median prime count and median prime amount calculations. For example, if there are less than nine episodes in the prior week, then the system 10 may access data for the most recent nine episodes regardless of predetermined time period to ensure that the data from a minimum of nine episodes is used in the medial prime count and median prime amount calculations.

In some implementations, the system 10 may determine the representative dose duration time value 138 using any of a variety of methods, including but not limited to calculating a representative dose duration time value 138 based on the user's recorded recent behavior (e.g., finding a median value, mean value, or selecting a value on a bell-curve distribution in the third or fourth quartile, etc.), prompting the user to select a dose duration time value to be used, finding a mean dose duration time value across a population of a subset thereof, relying on a default dose duration time value preselected in the software app 40, and the like. In the instant example, the system 10 determines the representative or typical dose duration time value 138 by selecting a dose duration value on a bell-curve distribution that is toward the higher end of typical for the user, such as a value in the third quartile (e.g. 51^{st} to 75th percentile) of dose duration values. By way of example, to determine the user representative dose duration time value 138, the system 10 calculates dose duration time value by subtracting the time value of the last dose from the time value of the first dose for all episodes within the predetermined prior time period. The system 10 then selects a value within the third quartile of the distribution of dose duration time values to be the representative dose duration time value. If there are less than a predetermined number of episodes in the predetermined time period, then the system 10 may access stored data for episodes occurring outside of (e.g. earlier than) the predetermined time period in order to include the predetermined number of episodes in the representative dose duration time value calculations. For example, if there are less than nine episodes in the prior week, then the system 10 may access data for the most recent nine episodes regardless of predetermined time period to ensure that the data from a minimum of nine episodes is used in the representative dose duration time value calculations.

The next step 140 is to determine the appropriate timing of recommendation dismissal. In some implementations, the system 10 may remove or dismiss the dose recommendation from the user interface screen 36 of the companion device 30 and/or pen device 20 when the system 10 determines that the sum value of the doses since the insulin recommendation time reaches or exceeds the following threshold:

(representative prime amount)*(representative prime count)+(insulin recommendation)−(dialing error correction value)

By way of example the dialing error correction value may vary depending on the increment size of the pen device 20 in use (e.g., 0.5 for a half-unit increment pen device, 1.0 for a one-unit increment pen device, etc.). In some implementations, the system 10 may remove or dismiss the dose recommendation from the user interface screen 36 of the companion device 30 and/or pen device 20 when the system 10 determines that the dose has a volume value greater than or equal to a predetermined number of units (e.g., 4 units). In some implementations, the system 10 may remove or dismiss the dose recommendation from the user interface screen 36 of the companion device 30 and/or pen device 20 when the system determines that the user's representative dose duration time from the previous week, and no more than two minutes, has passed since the time of the first dose. In some implementations, the system 10 may remove or dismiss the dose recommendation from the user interface screen of the companion device 30 and/or pen device 20 when the system determines that the insulin recommendation is 0.5, the user's median prime count=0, and the first positive-amount dose occurs. In some implementations, the system 10 may remove or dismiss the dose recommendation from the user interface screen of the companion device 30 and/or pen device 20 when the system 10 determines that the insulin recommendation is 0.5, the user's median prime count>0, and the second positive-amount dose occurs. In some implementations, the system 10 may remove or dismiss the dose recommendation from the user interface screen of the companion device 30 and/or pen device 20 when the system 10 determines that the user manually enters a dose value.

In some implementations, if a user manually changes a dose type to “prime”, the system 10 may re-display the dose recommendation on the user interface, exclude the dose manually changed to “prime,” and use one of the following to determine recommendation dismissal timing: (i) insulin recommendation—0.5, (ii) dose has a volume value greater than or equal to 4 units (for example), or (iii) the user's representative dose duration time from the previous week (and no more than two minutes) has passed since the time of the first dose.

By way of example, if the user has less than a predetermined total number of prior dosing episodes (e.g. nine), then the system 10 may display the dose recommendation on the user interface screen for up to a predetermined length of time (e.g. two minutes) after the start of the first positive-amount dose in the episode for a predetermined number of days (e.g., the first week of the user's episodes).

By way of example, if the system 10 identifies the episode as occurring after a cartridge replacement (e.g., the episode includes a retraction event) then the system 10 may display the dose recommendation on the user interface screen for up to a predetermined length of time (e.g. 60 seconds) from the most recent dose.

Confidence Scoring

In some implementations, the system 10 may use confidence scoring to determine whether a dose was therapeutic. By way of example, understanding the distributions of the users' prime behaviors can lead to a confidence scoring method for new dose interactions in the application. To do this, the system 10 may assign to each user a discrete univariate probability distribution with finite support describing each possible prime amount, and another describing each possible dose amount. From that probability distribution, the system 10 may calculate the probability of a given dose amount value being a prime dose amount. For example, if for a given user (e.g. a user with an inconsistent prime amount history), the median prime amount value is 1.0 U and the values of the user's primes are 0.5, 0.5, 1.0, 1.0, 1.5, 2.5, then the system 10 may calculate the probability of a dose amount having a value of 0.5 to be 33.33% likely to be a prime dose value. By way of example, a user who is more consistent with priming, may have a prime amount value distribution of 0.5, 0.5, 1.0, 1.0, 1.0, 1.0, 1.0, 1.0, 1.0, 1.0, 1.0, 1.0, 1.0, 1.0, 3.0. Thus for the consistent prime user, the system 10 may calculate the probability of a dose having a dose amount value of 1.0 U to be 80% likely to be a prime dose value. The system 10 may use these probabilities to generate “confidence scores” for prime dosing activity. In some embodiments, the system 10 may employ similar calculations to generate confidence scores for therapeutic dosing activity.

In some instances, other features such as paired recommendation amount may also influence the confidence score. In the above example, for a recommendation of 1.0 U, the confidence score would be lower as it is more difficult to distinguish.

For users that are highly predictable in their patterns and thus have high confidence scores, the system 10 may accurately designate a dose amount value as prime or therapeutic. However with some users with inconsistent methods, confidence scores may be lower, prompting the system 10 to implement one or more actions to mitigate the risk of recommendation dismissal in the absence of high confidence scores. In some implementations, the system may require a minimum confidence score to designate as a dose amount value to be therapeutic. In some implementations, the system 10 may establish a tunable indeterminate zone in between a confident therapeutic dose amount value threshold and confident prime dose amount value threshold. If confidence scores fall within the tunable indeterminate zone, the system 10 may implement one or more actions, including but not limited to displaying the dose recommendation on the user interface screen until a predetermined user episode duration (e.g., average, median, or a percentile value such as 90th) has passed, displaying the recommendation on the user interface screen 36 until a predetermined fixed amount of time has passed, dismissing the dose recommendation from the user interface screen 36, designating the dose as a therapeutic dose, designating the dose as a prime dose, displaying on the interface screen an indication of unrecognized dose type and prompting the user to designate the dose manually.

User Interactions

In some implementations, particularly for users that don't prime the pen device 20, or for inconsistent users that may cause inappropriate recommendation dismissals, the system 10 may flag the user as potentially benefiting from additional education, and activate an interactive training module to educate the user to practice consistent priming. For example, after a predetermined number of dosing episodes having no detected prime dose amount values, the system 10 may determine that the user is not priming, and display an interactive training module on the user interface screen 36. In some implementations, after the system 10 displays the interactive training module, a user who continues to decline to prime may be shown additional training modules (e.g. same as or different from the first training module) by the system 10 at predetermined intervals until the user modifies their behavior. In some implementations, after a defined number of re-educational interventions have failed to modify the user's behavior, the system 10 may initiate escalation to a care team member for human intervention. In some implementations, for users administering therapeutic doses with large dose amount values, and the resulting error from not utilizing a prime dose (e.g. less than 2 U) is a small portion of the therapeutic dose, the system 10 may avoid displaying the training module altogether since the user's priming technique may not greatly impact their therapy.

Multi-Part Dosing

In some implementations, the system 10 may update the dose recommendation in real time to inform users administering therapeutic doses over several injections. By way of example, for users who administer larger therapeutic doses in multiple injections, the system 10 may recognize the dose sequence as multiple smaller doses that add up to the total therapeutic dose. In this case, the system 10 may designate all doses actually injected as therapeutic, and update the dose recommendation in real time to display the dose amount value that is remaining from the initial dose recommendation that has not yet been injected (e.g. after injecting the first 30 units of a 50-unit dose recommendation, the system 10 may update the dose recommendation to instruct the user to administer the remaining 20 units of the original 50-unit dose recommendation).

By way of example, to accomplish this, the system 10 may first use the confidence score method described above to determine whether a dose was a prime dose. By way of example, if the confidence score for prime is above a predetermined threshold, the system 10 may recognize the dose as a prime dose and consequently would not update the dose recommendation amount. By way of example, if the confidence score for prime dose is low, the confidence score for therapeutic dose is high, or the administered dose is 4 units or greater, the system 10 may update the dose recommendation on the user interface screen to show the difference (original recommendation amount−dose amount).

In some instances, certain events may cause the pen injector 20 to dispense a small amount of medicine that is neither prime dose nor therapeutic dose. This may be referred to as a null event. For example, a null event may occur with a user conducting training on a new device, or inadvertently dispensing a small amount on the floor during handling, etc. In such an event, the system may be configured to flag these events as low confidence therapeutic doses. If the system 10 detects the possibility of a null event (e.g. system 10 detects emission of a small amount of medicine from injection pen 20), the system 10 may employ the confidence score method described above to automatically designate the dose as prime or therapeutic. By way of example, if the confidence score for both prime and therapeutic dose is low, the system may prompt the user for a manual designation or confirmation that the dose was null, prime, or therapeutic.

Data that the system 10 may include in the confidence analysis include (but are not limited to):
1. Glucose response after the dose—if the glucose values do not decrease as forecasted for a dose of the recorded size, and are better fit to the forecasted trend for the user taking no insulin, the confidence score may be low for therapeutic dose.
2. Timing—if the timing of the dose is atypical for that user, the confidence score may be lowered (e.g. user has no or low occurrence of dosing in the middle of the night, or the dose is too close to another therapeutic dose to be trusted as also being therapeutic).
3. Number of doses within a time—in the case where multiple dispense events are recorded in close proximity, all doses in the episode may be assigned a lowered confidence score.
4. Deviation from normal dosing pattern—the pattern of dose or doses in the sequence differs from the normal user pattern. The pattern can include the number and size of the prime dose, the timing between prime doses, the timing between the last prime dose and the last therapeutic dose in the sequence (the typical user dose).
5. User feedback from the system designation fed back into the confidence score analysis—if the system is categorizing therapeutic doses or prime doses incorrectly and the user is changing the categorizations manually, the overall confidence scoring analysis may be affected and the results may be biased in a direction to change the weight of the scoring such that historical results would be improved.
6. Additional sensor data available from the pen device 10 such as orientation, force, speed of delivery, absolute plunger position sensor, etc.

In some implementations, a combination of the above data points can be used in a time series anomaly detection system that indicates concern whenever the dose is significantly different from the otherwise normal predicted dose at that point in time.

In addition to the above methods for determining the timing of recommendation dismissal, the system 10 may be configured to display dose recommendations for a predetermined amount of time (e.g., 1 minute, 2 minutes, etc.) after every dose, such that the dose recommendation will be displayed past prime doses and until the therapeutic dose for most users. The system 10 may personalize this amount by measuring the episode duration from past dosing episodes as defined as the time from the first prime dose to the actual therapeutic dose (the last dose event in the sequence). Alternatively, the system may be configured to designate all doses having a dose amount value less than a predetermined dose amount value (e.g., less than 4 U) as prime initially, anticipating another dose to follow. If after a timeout, no dose is taken after the last dose, then the system 10 may change the designation of the last dose or doses in a multi-part dose episode from prime to therapeutic. The last dose or several doses is typically the therapeutic injected dose.

At least parts of the disclosed embodiments that include modules and the operations can be implemented in digital electronic circuitry, or in computer software, firmware, or hardware. At least some of those embodiments or operations can be implemented as one or more computer program products, i.e., one or more modules of computer program instructions encoded on a computer-readable medium for execution by, or to control the operation of, data processing apparatus. The computer-readable medium can be a machine-readable storage device, a machine-readable storage substrate, a memory device, a composition of matter affecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal, that is generated to encode information for transmission to suitable receiver apparatus.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method for determining an appropriate time duration to display a therapeutic dose recommendation associated with a current dispense event from a medicine dispensing device, comprising:
displaying, on a display device, a dose recommendation comprising a recommended therapeutic dose amount of medicine for a user of a medicine dispensing device;
analyzing use data pertaining to at least one of past and present dispense events for a particular user; and
removing the dose recommendation from the user interface based on the analysis of the use data.

2. The method of claim 1, wherein the use data comprises volume data from the last dispense event, and the volume of the last dispense event exceeds a preset threshold value.

3. The method of claim 1, wherein analyzing use data comprises:
identifying a set of episodes occurring within a predetermined period of time prior to the current episode, wherein an episode comprises a collection of prime dispensing activity and therapeutic dose dispensing activity occurring within a defined period of time following the display of the dose recommendation;
determining a representative prime count value based upon a count of prime events per episode within the identified set of episodes;
determining a representative prime amount value based upon a volume of dispensed medicine in prime events per episode within the identified set of episodes;
determining a representative dose duration time value based upon dose duration time values per episode within the identified set of episodes; and
calculating an appropriate timing for dose recommendation dismissal using at least one of the representative prime count value, the representative prime amount value, and the representative dose duration value.

4. The method of claim 3, wherein analyzing use data comprises:
calculating a sum value of dispense events occurring since the display of the dose recommendation, wherein a dispense event comprises any volume of medicine ejected from the medicine dispensing device; and
comparing the calculated sum value of dispense events to a user-specific threshold value.

5. The method of claim 4, wherein the user-specific threshold value is calculated using:
(representative prime amount)*(representative prime count)+(dose recommendation)−(dialing error correction value).

6. The method of claim 4, wherein the step of removing the dose recommendation from the user interface based on the analyzed use data comprises:
upon determining that the calculated sum value of dispense events at least one of meets and exceeds the user-specific threshold value, removing the displayed dose recommendation from the display device.

7. The method of claim 3, wherein the predetermined period of time comprises a length of time sufficient to establish a pattern of behavior of a user of the medicine dispensing device.

8. The method of claim 3, wherein the predetermined period of time comprises one week.

9. The method of claim 1, wherein analyzing use data pertaining to at least one of past and present dispense events for a particular user comprises
analyzing a set of past prime dispense events to calculate a confidence score that a given dispense event is a prime event; and
analyzing a set of past therapeutic dispense events to calculate a confidence score that a given dispense event is a therapeutic dose event.

10. The method of claim 9, wherein the step of removing the dose recommendation from the user interface based on the analyzed use data comprises:
upon determining that the current dispense event has a volume that at least one of meets and exceeds a preset confidence score threshold value, removing the displayed dose recommendation from the display device.

11. The method of claim 1, wherein the display device comprises at least one of the medicine dispensing device and a mobile communication device associated with the medicine dispensing device.

12. A method for differentiating between a therapeutic dose and a priming event in dispensing medication by a medication dispensing device, comprising:
analyzing a plurality of dispensing events that occur in a sequence, the sequence including at least a first dispensing event and a last dispensing event, each dispensing event comprising a dispensed amount of medication from the medication dispensing device;
classifying at least one of the dispensing events occurring prior to the last dispensing event as a therapeutic dose dispensing event based on at least one of:
(a) the dispensed amount has a volume that is above a predefined threshold volume;
(b) the dispensed amount has a volume that is equal to a maximum volume capacity of the dispensing device; and
(c) the dispensed amount exhausts the supply of medicine contained within the dispensing device.

13. A method for differentiating therapeutic and priming events in administering a medication by a medication injection device, comprising:
analyzing a plurality of dispensing events that occur in a sequence within a preset time period and within a dose window; and
classifying at least one of the dispensing events as a therapeutic dose dispensing event based on one or more criteria.

14. The method of claim 13, comprising:
estimating a duration of a single dispensing event in the sequence to differentiate a single dispensing event from multiple consecutive dispensing events in the sequence using a variable time threshold; and
deriving a time window based on the duration of the single dispense event.

15. The method of claim 14, wherein the variable time threshold is adjusted based on past dispensing events of a user of the medicine injection device.

16. The method of claim 14, wherein the variable time threshold is adjusted based on a speed of movement when a user manipulates the medication injection device for dispensing a therapeutic dose or dispensing a prime dose.

17. The method claim 13, wherein the classifying comprises:
designating a dispensing event as the therapeutic dose dispensing event upon determining that an amount of the medication released in the dispensing event exceeds a predefined threshold.

18. The method of claim 17, wherein the predefined threshold is determined based on past dispensing events of the patient.

19. The method of claim 13, wherein the classifying comprises:
designating a dispensing event by comparing an amount of the medication released in the dispensing event with a predefined priming dosage amount.

20. The method of claim 13, wherein the classifying comprises:
designating a dispensing event as a priming event upon determining that the dispensing event occurs immediately after a cartridge replacement of the medication injection device.

21. The method of claim 13, wherein the classifying comprises:
designating a first dispensing event in the sequences as the therapeutic dose dispensing event upon determining that an amount of the medication released in a first dispensing event reaches a maximum limit of the medication injection device;
designating a second dispensing event immediately following the first dispensing event as the therapeutic dose dispensing event; and
accumulating the amount of the medication released in the first and second dispensing events for the calculation and tracking of a dosage amount for a patient.

22. The method of claim 13, wherein the classifying comprises:
notifying a user when there is an ambiguity in the classifying of a dispensing event; and
receiving an input from the user to confirm whether the dispensing event is a therapeutic dose dispensing event or a priming event.

23. An apparatus for differentiating a therapeutic dispensing event and a priming event in administering a medication, comprising:
a processor, and
a non-transitory memory including processor executable code, wherein the processor executable code upon execution by the processor configures the processor to:
determine, based on an input from a sensor of an injection device, whether the injection device is in contact with a patient or is held freely;
designate a dispensing event as a priming event when the device is held freely; and
designate a dispensing event as a dosing event when the device is in contact with a patient.

24. The apparatus of claim 23, wherein the sensor of the injection device includes at least one of an accelerometer, a pressure sensor and a vibration sensor.

25. The apparatus of claim 23, wherein the sensor of the injection device includes a capacitive sensor coupled to a needle of the injection device to detect a skin contact electrically.

26. The apparatus of claim 23, wherein the sensor of the injection device includes a depth sensor or a capacitive sensor oriented toward an end of a needle of the injection device to detect a nearby surface.

27. The apparatus of claim 23, wherein the processor executable code upon execution by the processor configures the processor to:
activate, prior to detecting a priming event, a locking mechanism on the injection device upon determining that the injection device is not oriented vertically upward; and
deactivate the locking mechanism after a priming event is detected.

28. The apparatus of claim 23, wherein the injection device is configured to perform the first dispense of the medication as the priming event automatically.
